(19) United States
(12) Patent Application Publication
(10) Pub. No.: US 2026/0105464 A1
(43) Pub. Date: Apr. 16, 2026

(54) BIOLOGICAL DATA PREDICTION SYSTEM FOR TREES

(71) Applicant: CATALONIX INC., Chuncheon-si, Gangwon-do (KR)

(72) Inventors: Min SUK, Seoul (KR); Kyung Doe HAN, Chuncheon-si, Gangwondo (KR)

(73) Assignee: CATALONIX INC., Chuncheon-si, Gangwon-do (KR)

(21) Appl. No.: 19/114,486

(22) PCT Filed: Nov. 15, 2022
(86) PCT No.: PCT/KR2022/017937

§ 371 Date: Mar. 24, 2025

(30) Foreign Application Priority Data
Nov. 10, 2022 (KR) 10-2022-0149311

(51) Int. Cl. G06Q 30/018; G16B 40/00

(52) U.S. Cl. CPC G06Q 30/018; G16B 40/00

(57) ABSTRACT

The present invention relates to a biological data prediction system for trees, wherein the biological data prediction system includes: a three-dimensional tree observation data collection part configured to collect three-dimensional tree observation data in a predetermined zone; a two-dimensional tree observation data collection part configured to collect two-dimensional tree observation data in the predetermined zone; an environmental data collection part configured to collect environmental data in the predetermined zone; and a biological data prediction part configured to predict biological data of the trees from the three-dimensional tree observation data, the two-dimensional tree observation data, and the environmental data.

### FIELD OF THE INVENTION

The present disclosure relates to a biological data prediction system for trees. More particularly, the present disclosure relates to a system for predicting biological data of trees from three-dimensional tree observation data, two-dimensional tree observation data, and environmental data for the trees in a predetermined zone.

### BACKGROUND ART

In the photosynthesis process, trees absorb gaseous substances such as carbon dioxide, sulfur dioxide, and nitrogen dioxide, which are greenhouse gases, through pores of leaves, thereby lowering atmospheric concentrations of the gaseous substances and providing fresh air while emitting oxygen required for human respiration.

Such an air purification role of trees has been studied from various perspectives, such as experimental and physiological, but most of the studies have been conducted in a laboratory or a given environment, so that there is almost no biological data on trees little biological data on trees in a natural state.

Conventionally, tree growth information, environmental information, and disease and insect information were mainly used to predict the production of crops, and biological data of trees, i.e., a photosynthesis amount, a carbon dioxide capture amount, a fine dust capture amount, and so on, were not predicted through the growth information, the environmental information, and so on, so that there was no method for knowing how much trees can respond to a change in the environment.

### DISCLOSURE OF INVENTION

### Technical Problem

Accordingly, the present disclosure has been made keeping in mind the above problems occurring in the related art, and an objective of the present disclosure is to provide a system for predicting biological data of trees in a predetermined zone from three-dimensional tree observation data, two-dimensional tree observation data, and environmental data.

The objectives that can be obtained from the present disclosure are not limited to the above-mentioned objectives, and other objectives not mentioned herein will be clearly understood by those skilled in the art from the following description.

### Technical Solution

In order to achieve the above objective, according to the present disclosure, there is provided a biological data prediction system for trees, the biological data prediction system including: a three-dimensional tree observation data collection part configured to collect three-dimensional tree observation data in a predetermined zone; a two-dimensional tree observation data collection part configured to collect two-dimensional tree observation data in the predetermined zone; an environmental data collection part configured to collect environmental data in the predetermined zone; and a biological data prediction part configured to predict biological data of the trees from the three-dimensional tree observation data, the two-dimensional tree observation data, and the environmental data.

Here, the three-dimensional tree observation data collection part may include at least one selected from: a ground stationary three-dimensional measurement part fixed to a ground and configured to collect three-dimensional tree observation data in the predetermined zone while being in a stationary state; an aerial mobile three-dimensional measurement part disposed on an aerial portion of the predetermined zone such that the aerial mobile three-dimensional measurement part is in a mobile state, the aerial mobile three-dimensional measurement part being configured to collect three-dimensional tree observation data in the aerial portion of the predetermined zone while being in the mobile state; and a ground mobile three-dimensional measurement part disposed on the ground of the predetermined zone such that the ground mobile three-dimensional measurement part is in the mobile state, the ground mobile three-dimensional measurement part being configured to collect three-dimensional tree observation data in the predetermined zone while being in the mobile state.

In addition, the two-dimensional tree observation data collection part may include a multispectral camera.

In addition, the environmental data collection part may include: a soil environmental data collection part configured to collect soil environmental data in the predetermined zone; and an atmospheric environmental data collection part configured to atmospheric environmental data in the predetermined zone.

In addition, the biological data prediction part may include a three-dimensional vegetation index calculation part configured to calculate a three-dimensional vegetation index of the trees in the predetermined zone.

Here, the biological data prediction part may further include a three-dimensional growth amount calculation part configured to calculate a three-dimensional growth amount of the trees in the predetermined zone.

In addition, the biological data prediction part may further include a carbon dioxide capture amount calculation part configured to calculate a carbon dioxide capture amount of the trees in the predetermined zone.

In addition, the biological data prediction part may further include a fine dust capture amount calculation part configured to calculate a fine dust capture amount of the trees in the predetermined zone.

### Advantageous Effects

According to the biological data prediction system for trees according to the present disclosure, there are the following effects.

In the biological data prediction system for trees according to the present disclosure, there is an advantage that calculation of the planting amount and the maintenance of the trees are capable of being performed easily since the biological data for trees are calculated or predicted when the three-dimensional data of the trees, the two-dimensional data of the trees, and the environmental data are input in the biological data prediction system.

In addition, in the biological data prediction system for trees according to the present disclosure, the biological data such as the carbon dioxide capture amount, the fine dust capture amount, and so on are capable of being predicted by using a single physical quantity such as the vegetation index, so that there is an effect that the prediction cost is capable of being reduced and also the time for performing the prediction is capable of being reduced.

Effects of the present disclosure are not limited to the effects mentioned above, and other effects not mentioned will be clearly understood by those skilled in the art from the description of the claims.

### BRIEF DESCRIPTION OF THE DRAWINGS

FIG. 1 is a schematic view illustrating an overall configuration of a biological data prediction system for trees according to an embodiment of the present disclosure.

FIG. 2 is a view illustrating configurations of a three dimensional tree observation data collection part, a two-dimensional tree observation data collection part, an environmental data collection part, and a biological data prediction part f the biological data prediction system for trees according to an embodiment of the present disclosure.

FIG. 3 is a view illustrating a configuration of a vegetation index acquisition unit according to another embodiment of the present disclosure.

FIG. 4 shows exemplary views illustrating an example of acquiring a first image by the vegetation index acquisition unit in FIG. 3.

FIG. 5 shows exemplary views illustrating an example of acquiring a second image by the vegetation index acquisition unit in FIG. 3.

FIG. 6 shows images showing a two-dimensional first vegetation index and a disparity map acquired by the vegetation index acquisition unit in FIG. 3.

FIG. 7 is an exemplary view illustrating image synchronization and mapping in the vegetation index acquisition unit in FIG. 3.

FIG. 8 is a view illustrating a configuration of the vegetation index acquisition unit according to still another embodiment of the present disclosure.

FIG. 9 is an exemplary view illustrating a vegetation index acquisition example of the vegetation index acquisition unit in FIG. 8.

FIG. 10 is a view schematically illustrating the configuration of the biological data prediction part illustrated in FIG. 2.

FIG. 11 shows views illustrating a calculation method of a three-dimensional vegetation index calculation part.

FIG. 12 shows views illustrating a calculation method of a three-dimensional growth amount calculation part.

FIG. 13 shows views illustrating a calculation method of a carbon dioxide capture amount calculation part.

FIG. 14 shows views illustrating a calculation method of a fine dust capture amount calculation part.

FIG. 15 is a view schematically illustrating a configuration of the biological data prediction part of the biological data prediction system for trees according to still another embodiment of the present disclosure.

FIG. 16 is a view illustrating a prediction method of a three-dimensional growth amount prediction part illustrated in FIG. 15.

FIG. 17 is a view illustrating a prediction method of a carbon dioxide capture amount prediction part illustrated in FIG. 15.

FIG. 18 is a view illustrating a prediction method of a fine dust capture amount prediction part illustrated in FIG. 15.

### DETAILED DESCRIPTION OF THE INVENTION

Advantages and features of the present disclosure, and methods of achieving the same will become apparent with reference to the embodiments described below in detail in conjunction with the accompanying drawings. However, the present disclosure is not limited to the embodiments disclosed below, but may be implemented in various different forms. The present embodiments are intended to complete the disclosure of the present disclosure and provided to fully inform the skilled in the art to which the disclosure pertains of the scope of the disclosure. The disclosure is defined only by the scope of the claims. Like reference numerals indicate like components throughout the specification.

In addition, the sizes, the shapes, and so on of components illustrated in the drawings may be exaggerated for convenience of understanding. In addition, it should be noted that the same components may be denoted by the same reference numerals throughout the specification. In addition, a detailed description of known functions and configurations incorporated herein may be omitted when it may obscure the subject matter of the present disclosure.

The terminology used herein is for the purpose of describing particular embodiments and is not intended to be limiting. As used in this specification, a singular form may include a plural form unless definitely indicating a particular case in terms of the context. Throughout the present specification, when a part is referred to as “including” an element, it means that the part may include other elements as well without excluding the other elements unless specifically stated otherwise.

When a component is described as being “connected”, “coupled”, or “linked” to another component, that component may be directly connected, coupled, or linked to that other component. However, it should be understood that yet another component between each of the components may be present. On the other hand, when a component is referred to as being “directly connected” or “directly contacted” to another component, it should be understood that there is no other component therebetween. Other expressions for explaining the relationship between components should be interpreted in the same manner.

Terms such as the upper end, the lower end, the upper surface, the lower surface, the upper part, the lower part and so on used in the present specification are used to distinguish relative positions in components. For example, when the upper part of the drawing is referred to as the upper part and the lower part of the drawing is referred to as the lower part for convenience, the upper part may be referred to as the lower part and the lower part may be referred to as the upper part without departing from the scope of the present disclosure.

Terms including ordinal numbers, such as “a first”, “a second”, and so on described in the present specification, may be used to describe various components, but the components are not limited thereto. The terms are only referred to in order to distinguish that each component is different, are not limited to the order of manufacture, and the names thereof may not match in the detailed description of the present disclosure and the claims.

All terms including technical or scientific terms used herein have the same meaning as those generally understood by those skilled in the art to which the present disclosure belongs unless otherwise defined. Terms such as those defined in commonly used dictionaries should be interpreted as having meaning consistent with the contextual meaning of the relevant technology and are not interpreted as ideal or excessively formal unless clearly defined in this specification.

Hereinafter, in order to describe a biological data prediction system for trees according to an embodiment of the present disclosure, the present disclosure will be described with reference to the drawings.

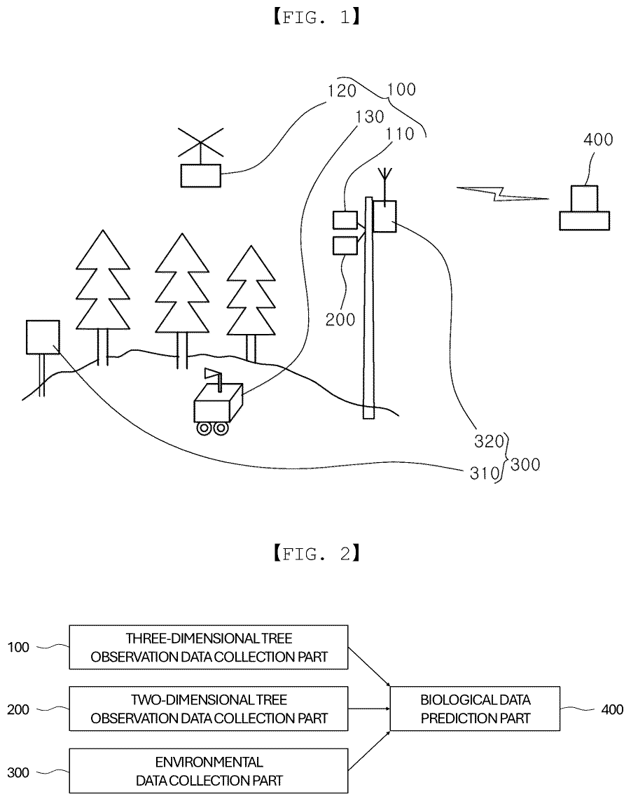

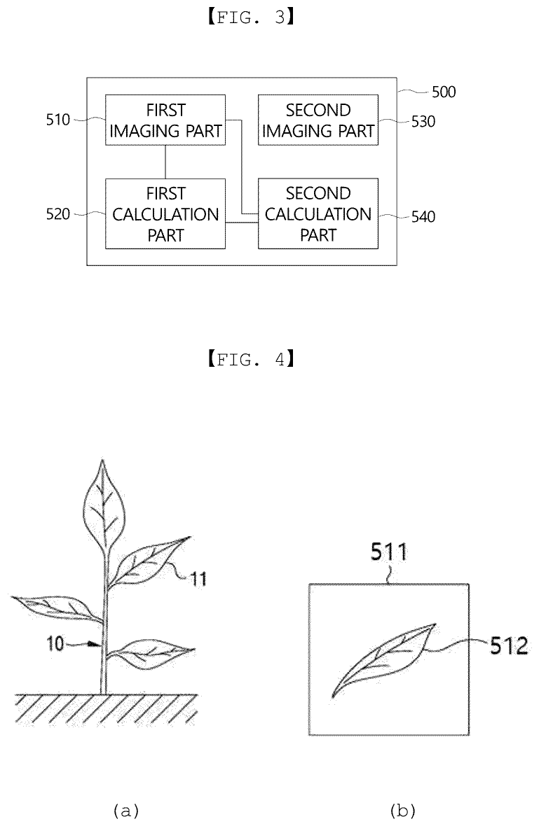

Referring to FIG. 1 and FIG. 2, the biological data prediction system for trees according to an embodiment of the present disclosure may include a three-dimensional tree observation data collection part 100, a two-dimensional tree observation data collection part 200, an environmental data collection part 300, and a biological data prediction part 400.

The three-dimensional tree observation data collection part 100 uses a measurement device such as a stereo camera, LiDAR, or a radar and observes and collects data on trees in a predetermined zone by being moved through air on the ground, and may collect observation data such as RGB color information, a 3D model of a tree object, and metadata (tree information, observation date and time, a location) in a three-dimensional form.

The two-dimensional tree observation data collection part 200 uses a device such as a near-infrared signal analysis camera or a multispectral camera and observes and collects data on the trees in the predetermined zone from a fixed location in the air or on the ground, and may collect observation data such as RGB, NIR, a vegetation index, a dryness, and a water stress index of trees in the predetermined zone in a two-dimensional form.

The environmental data collection part 300 includes a soil sensor or a weather sensor and collects environmental data such as atmosphere data and soil data in the predetermined zone, and may measure and collect data such as a sunlight amount, a temperature and humidity, a precipitation amount, fine dust data, a carbon dioxide amount, a soil structure, and a soil moisture content for the predetermined zone.

The biological data prediction part 400 is configured to predict biological data of the trees in the predetermined zone through machine learning. The biological data prediction part may predict biological data such as a vegetation index, a growth amount, a carbon dioxide capture amount, and a fine dust capture amount of the trees in the predetermined zone by performing the machine learning matching the RGB color information, the 3D model of the tree object, and the metadata (the tree information, the observation date and time, the location) of the trees in the predetermined zone collected by the three-dimensional tree observation data collection part 100, the RGB, the NIR, the vegetation index, the dryness, and the water stress index of the trees in the predetermined zone collected by the two-dimensional tree observation data collection part 200, and the sunlight amount, the temperature and humidity, the precipitation amount, the fine dust data, the carbon dioxide amount, the soil structure, and the soil moisture content collected by the environmental data collection part 300 to each other.

Here, the three-dimensional tree observation data collection part 100 may include at least one selected from a ground stationary three-dimensional measurement part 110, an aerial mobile three-dimensional measurement part 120, and a ground mobile three-dimensional measurement part 130.

The ground stationary three-dimensional measurement part 110 is fixed to the ground in the predetermined zone and collects observation data on the trees in the predetermined zone in the three-dimensional form. Furthermore, a measurement device such as a stereo camera, LiDAR, or a radar may be mounted at a predetermined height on a streetlight, a pole, a signpost or the like mounted on the ground in the predetermined zone, and may collect observation data such as RGB color information, a 3D model of a tree object, and metadata (tree information, observation date and time, a location) of the trees in the predetermined zone in a three-dimensional image form.

The aerial mobile three-dimensional measurement part 120 is disposed on an aerial portion of the predetermined zone, and is configured to collect aerial observation data on the trees in the predetermined zone. Furthermore, the aerial mobile three-dimensional measurement part 120 may use a flight device such as an unmanned drone or a multicopter provided with a measurement device such as a stereo camera, LiDAR, or a radar, and may collect observation data such as RGB color information, a 3D model of a tree object, and metadata (tree information, observation data and time, a location) from an aerial portion of the trees in the predetermined zone where the ground stationary three-dimensional measurement part 110 is not capable of performing measurement.

The ground mobile three-dimensional measurement part 130 is disposed on the ground in the predetermined zone such that the ground mobile tree-dimensional measurement part 130 is capable of being moved on the ground, and is configured to be moved and to collect observation data on the trees in the predetermined zone in a three-dimensional form. Furthermore, the ground mobile three-dimensional measurement part 130 may use a ground driving device such as an unmanned robot or a vehicle provided with a measurement device such as a stereo camera, LiDAR, or a radar, and may collect observation data such as RGB color information, a 3D model of a tree object, and metadata (tree information, observation data and time, a location) from a lower portion of the trees in the predetermined zone where the ground stationary three-dimensional measurement part 110 and the aerial mobile three-dimensional measurement part 120 are not capable of performing measurement.

### In Addition, the Two-dimensional Tree Observation Data collection part 200 may include a near-infrared signal analysis Camera or a multispectral camera.

The near-infrared signal analysis camera is configured to analyze a near-infrared signal of the trees in the predetermined zone, and the multispectral camera is configured to photograph the trees in the predetermined zone at a specific wavelength range. Furthermore, the near-infrared signal analysis camera and the multispectral camera may be mounted at a predetermined height on a pole such as a telephone pole, a streetlight, or a signpost fixed to the ground and provided in the predetermined zone, and may collect observation data such as RGB, NIR, a vegetation index, a dryness, and a water stress index of the trees in the predetermined zone in a two-dimensional form.

Here, as another embodiment of the present disclosure, the three-dimensional tree observation data collection part 100 and the two-dimensional tree observation data collection part 200 may include a vegetation index acquisition unit 500, and may collect observation data of the trees in the predetermined zone in two-dimensional and three-dimensional forms.

Referring to FIG. 3, in the vegetation index acquisition unit 500, the two-dimensional tree observation data collection part 200 may be a first imaging part 510 and a first calculation part 520, and the three-dimensional tree observation data collection part 300 may be a second imaging part 530 and a second calculation part 540.

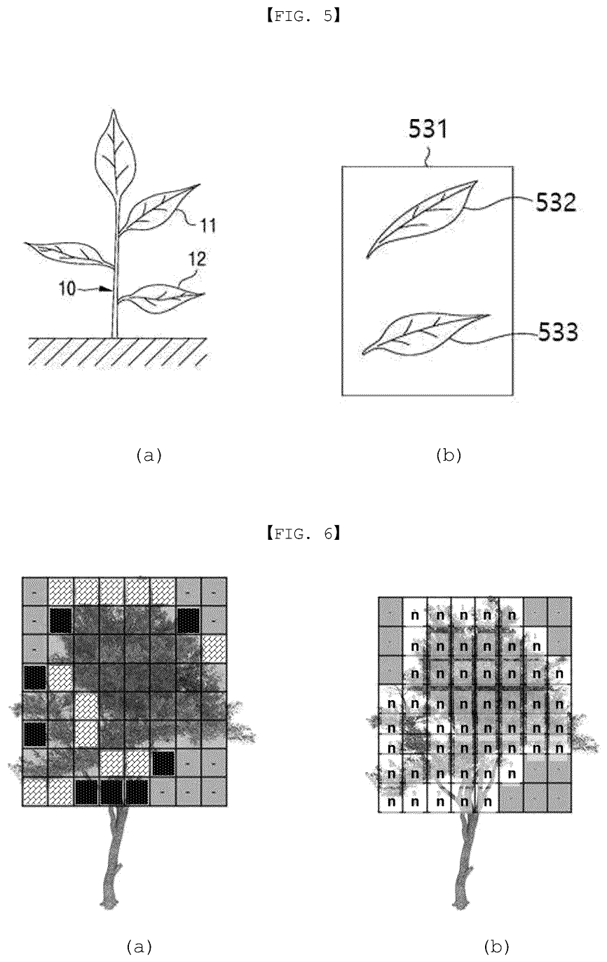

Referring to FIG. 4, exemplary views illustrating an example in which the vegetation index acquisition unit 500 acquires a first image are illustrated. The first imaging part 510 may acquire a first image 511 by photographing a first leaf 11 of a vegetation 10. Here, the first leaf 11 may be any one leaf selected from various leaves of the vegetation 10, and is not specifically limited.

The first image 511 may be a two-dimensional image. The first image 511 may include image information of a two-dimensional first leaf 512 that is generated by photographing the actual first leaf 11.

The first imaging part 510 is not specifically limited in type as long as the first imaging part 510 is capable of photographing the first image 511. As an example, the first imaging part 510 may include a near-infrared signal analysis camera, a multispectral camera, and so on.

The first calculation part 520 may calculate a first vegetation index 521 on the basis of the image information of the two-dimensional first leaf 512 contained in the first image 511. The first vegetation index 521 may be a Normalized Difference Vegetation Index (NDVI).

Since the first image 511 is a two-dimensional image, processing of the image information of the two-dimensional first leaf 512 may be accurately performed, that accuracy of calculating the first vegetation index 521 may be increased.

In FIG. 4, the first leaf 11 is illustrated as a single leaf, but this is an example and a plurality of leaves may be selected as the first leaf 11. When the first leaf 11 is a plurality of leaves, the first vegetation index 521 may be calculated for each leaf.

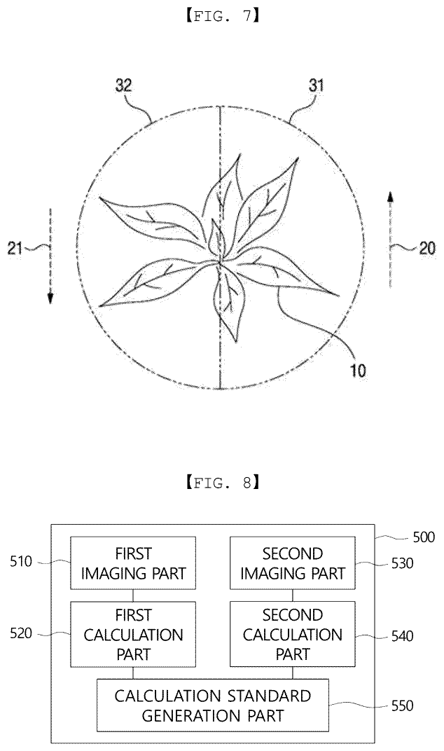

FIG. 5 shows exemplary views illustrating an example of acquiring a second image by using the vegetation index acquisition unit 500.

The second imaging part 530 may acquire a second image 531 by photographing another leaf (hereinafter, referred to as a second leaf 12) of the vegetation 10 together with the first leaf 11 of the vegetation 10 photographed by the first imaging part 510.

In FIG. 5, the second leaf 12 is illustrated as a single leaf, but this is an example and the second leaf 12 may collectively refer to all leaves except the first leaf 11. Therefore, the second image 531 may be a photograph of all the leaves of the vegetation 10.

The second image 531 may be a three-dimensional image. That is, the second imaging part 530 may photograph a three-dimensional shape of the vegetation 10, and the three-dimensional shape of the vegetation 10 may be restored in the second image 531.

The second imaging part 530 may include an imaging device capable of generating a three-dimensional image. for example, the imaging device may be a stereo camera.

The second calculation part 540 may calculate a second vegetation index 541 of the second leaf 12 by using the first vegetation index 521 for the first leaf 11, the first vegetation index 521 being calculated by the first calculation part 520.

Specifically, the second calculation part 540 may compare a difference value between image information of a three-dimensional first leaf 532 in the second image 531 and image information of the two-dimensional first leaf 512 in the first image 511. In addition, when the difference value is within a predetermined standard difference value, the second vegetation index 541 of the three-dimensional first leaf 532 in the second image 531 may be calculated in the same manner as the first vegetation index 521.

In addition, the second calculation part 540 may compare the difference value between image information of the three-dimensional first leaf 532 in the second image 531 and image information of the two-dimensional first leaf 512 in the first image 511. Furthermore, when the difference value is within the predetermined standard difference value, the second vegetation index 541 of the three-dimensional second leaf 533 in the second image 531 may be calculated in the same manner as the first vegetation index.

That is, in a situation in which the second calculation part 540 synchronizes a vegetation index image and a stereo image, when a combination of the vegetation index image and the stereo image in data already measured is referred to as A and a channel-specific signal (for example, simple RGB or CMYK without distance information) of a stereo image of a portion in which a vegetation index thereof is not measured is very similar, the second calculation part 540 may include a vegetation index of A in the unmeasured portion.

In addition, the second calculation part 540 may perform interpolation on the basis of a plurality of second vegetation indices already calculated and generated and image information of a second leaf of the plurality of second vegetation indices, thereby being capable of generating a second vegetation index for any other second leaf in which a first vegetation index thereof is not calculated.

FIG. 6 shows images showing a two-dimensional first vegetation index and a disparity map acquired by the vegetation index acquisition unit according to a first embodiment of the present disclosure.

As illustrated in FIG. 6, a reference having existing accurate position information, reflectivity information, and size information may be set within a photographing angle of view at the beginning of measurement so that and a benchmark is set, and trees and vegetation may be photographed centered around the benchmark simultaneously with the start of measurement. At this time, it is preferable that the first imaging part and the second imaging part are mounted in the same axis line, so that image synchronization may be stably performed.

FIG. 6(a) is a measurement illustrating a measured vegetation index in a two-dimensional plane, and FIG. 6(b) is a two-dimensional plane illustrating a disparity map acquired by the second imaging part. The difference in resolution of the measurement image in FIG. 6(a) and the disparity map in FIG. 6(b) is corrected for the angle of view along with the upscale and the downscale, so that the image may be synchronized. Since distance information is capable of being acquired through the disparity map acquired through the second imaging part, the distance information may be converted into a point cloud through an appropriate algorithm.

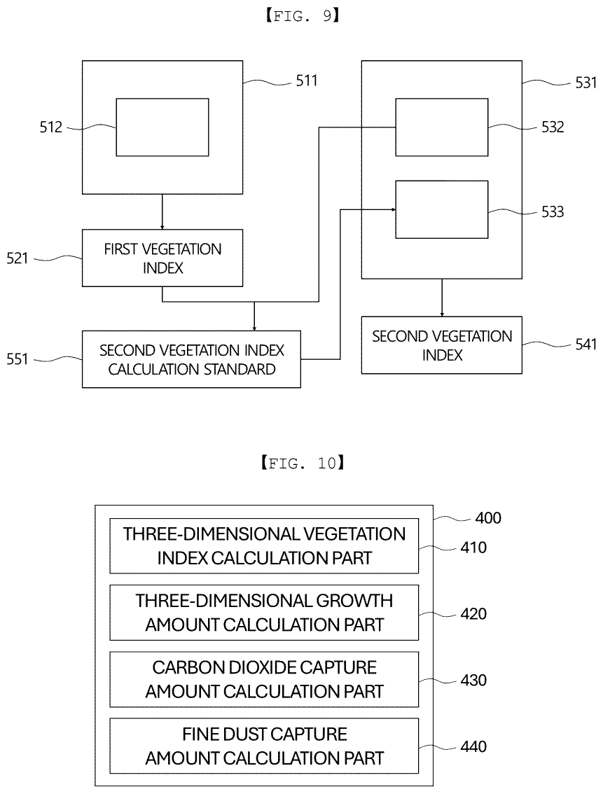

FIG. 7 is an exemplary view illustrating image synchronization and mapping in the vegetation index acquisition unit according to another embodiment of the present disclosure.

The vegetation is photographed by moving the first imaging part and the second imaging part around the vegetation, and the point cloud through three-dimensional imaging is synchronized with the position of the leaf on which the vegetation index is photographed or the pixel position of the disparity map is synchronized, so that the three-dimensional vegetation index image is acquired, thereby being capable of constructing the three-dimensional vegetation index.

Specifically, as illustrated in FIG. 7, in a situation in which a virtual vegetation 10 is assumed as a spherical shape, when a right hemisphere 31 of the vegetation 10 is measured by performing the measurement toward a right movement path 20 of the vegetation 10 and then a left hemisphere 32 is measured by performing the measurement toward a left movement path 21 is moved to the right movement path 20 of the vegetation 10, the converted point cloud is interlocked with a driving distance of the measurement so that a left measurement point cloud and a right measurement point cloud are combined at each position where the vegetation 10 is measured, thereby being capable of completing the 3D model.

In synchronizing the left and right images, a method of counting the number of measured vegetations 10 by using the average distance measured from the depth image (recognizing the presence or absence of vegetations when creating a close-up image) and an algorithm that recognizes the same vegetations 10 when the vegetations 10 are measured from different directions using GPS coordinates may be applied simultaneously. For example, when an image of a first vegetation and a second vegetation is recorded, the image may be stored as [2, 1]. Furthermore, when an image of the second vegetation and a second vegetation from the end is recorded, the image may be stored as [2, 2] so that lexicon is unified. Through this, 3D shape information for each vegetation may be provided.

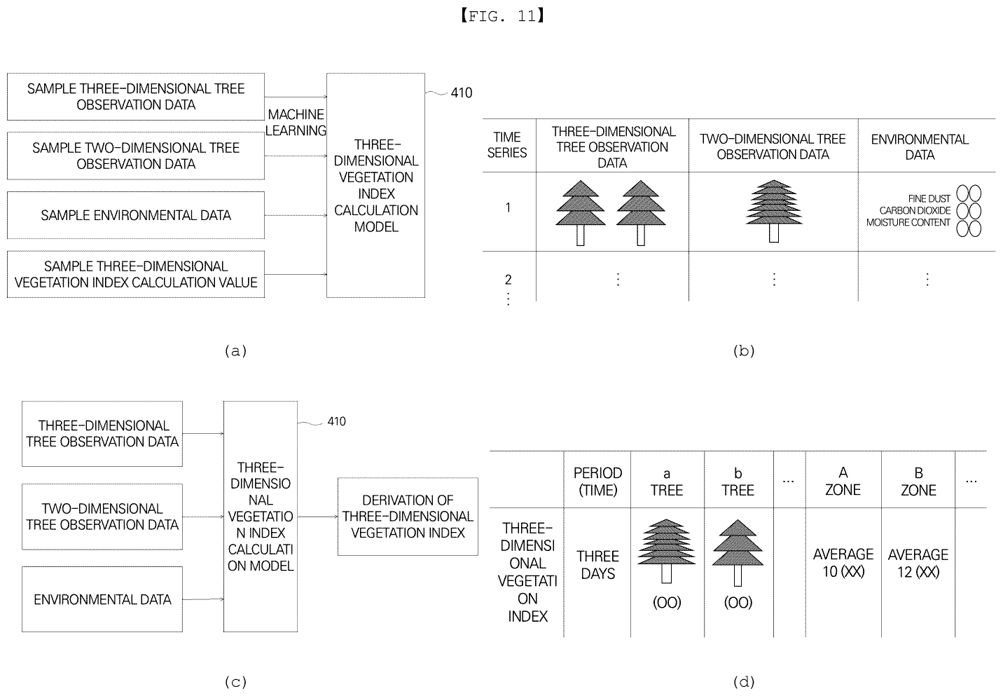

Meanwhile, FIG. 8 is a view illustrating a configuration of the vegetation index acquisition unit according to still another embodiment of the present disclosure, and FIG. 9 is an exemplary view illustrating a vegetation index acquisition example of the vegetation index acquisition unit in FIG. 8. In the present embodiment, a second vegetation index calculation standard is generated and the second vegetation index may be calculated by using the second vegetation index calculation standard, and other basic contents may be similar to the first embodiment described above.

In addition to FIG. 4 and FIG. 5, as illustrated in FIG. 8 and FIG. 9, a vegetation index acquisition unit 500 according to the

### Present Embodiment May Further Include a Calculation Standard generation part 550.

The calculation standard generation part 550 may generate a second vegetation index calculation standard 551 by matching the image information for the three-dimensional first leaf 532 included in the second image 531 and the first vegetation index 521.

The image information of the two-dimensional first leaf 512 in the first image 511 and the image information the three-dimensional first leaf 532 in the second image 531 may include color information, luminance information, and so on. However, since the second image 531 is a three-dimensional image, the image information of the three-dimensional first leaf 532 in the second image 531 may be different from the image information of the two-dimensional first leaf 512 in the first image 511. Therefore, when the vegetation index of the actual first leaf 11 is calculated by using the image information of the three-dimensional first leaf 532 in the second image 531, the second vegetation index calculated at this time may be different from the first vegetation index 521 calculated previously.

In order to solve this problem, in the present disclosure, the calculation standard generation part 550 may generate the second vegetation index calculation standard 551 by matching the image information of the three-dimensional first leaf 532 in the second image 531 and the first vegetation index 521 that is calculated on the basis of the image information of the two-dimensional first leaf 512 in the first image 511.

When the generated second vegetation index calculation standard 551 is applied to the second image, the second vegetation index may be calculated in a second image state, and the second vegetation index calculated in this manner may be the same as the first vegetation index.

The calculation standard generation part 550 generates the second vegetation index calculation standard 551, applies the generated second vegetation index calculation standard 551 to the image information of the three-dimensional first leaf 532 in the second image 531, and checks whether the second vegetation index calculated accordingly is the same as the first vegetation index 521. Furthermore, when the calculated second vegetation index is not the same as the first vegetation index 521, a process of correcting the second vegetation index calculation standard 551 is repeated, so that the accuracy of the generated second vegetation index calculation standard may be further increased.

The method in which the calculation standard generation part 550 generates the second vegetation index calculation standard 551 is not specifically limited.

The second calculation part 540 may apply the second vegetation index calculation standard 551 to the image information for the three-dimensional second leaf 533 in the second image 531, and may calculate the second vegetation index 541 of the actual second leaf 12 through this.

When the actual first leaf 11 is used as an example, the second vegetation index calculated for the three-dimensional first leaf 532 of the second image 531 may be the same value or a range value as the first vegetation index 521 calculated on the same actual first leaf 11.

As described above, since the second leaf 12 may collectively refer to one or more leaves except for the first leaf 11, the first leaf 11 and the second leaf 12 may refer to all leaves of the vegetation 10. Therefore, the second vegetation index 541 calculated may be for almost all leaves of the vegetation 10, so that a three-dimensional second vegetation index calculation of almost all leaves of the vegetation 10 is capable of being realized.

Generally, it is difficult to photograph an image or to receive a near-infrared signal in a portion such as, for example, an opposite portion of a vegetation where the first imaging part 510 cannot perform photographing, so that it is difficult to calculate the first vegetation index in the portion. However, when the first vegetation index in a portion such as a front side of the vegetation where the first imaging part 510 can perform photographing is calculated by photographing the portion and receiving a near-infrared signal and then the second imaging part 530 acquires the three-dimensional shape image information of the vegetation, the second vegetation index calculation standard 551 is generated, and a second vegetation index in the opposite portion of the vegetation is capable of being generate by using the second vegetation index calculation standard 551.

Alternatively, the second vegetation index may be generated by generating the second vegetation index calculation standard on the basis of the first vegetation index acquired by photographing a first portion (for example, an upper surface) of a leaf and then by including the second vegetation index calculation standard in the second image in which a lower surface of the leaf is photographed.

Alternatively, the second vegetation index may be generated by interpolating an overall measurement value for an object such as coniferous trees difficult to pixelize.

In a situation in which the vegetation 10 belongs to the category of leafy vegetables having edible leaves and including Ligularia stenocephala, Chrysanthemum coronarium, Aster scaber, Amaranthus, Petasites japonicus, and so on, when a simple prediction in which a vegetation condition of remaining leaves will be the same on the basis of a vegetation condition of one leaf is performed, maintaining the quality of harvested leafy vegetables may be difficult to be performed.

That is, when the vegetation index of the remaining leaves is predicted on the basis of the vegetation index of some leaves, the vegetation index of the remaining leaves may be affected according to the base vegetation index. For example, when the vegetation index of some leaves is determined to be good, it can be predicted that the vegetation index of the remaining leaves will also be good. However, since the vegetation may have different growth states for each part, the accuracy of this vegetation index prediction method may be low. However, when the vegetation index is calculated by photographing each leaf one by one, a considerable amount of time may be required.

However, according to the present disclosure, since the three-dimensional shape of the vegetation is photographed and the second vegetation index is capable of being easily and accurately calculated for all leaves, accurate and rapid monitoring of the vegetation is capable of being realized.

Meanwhile, although it is described earlier that the second vegetation index is calculated on the leaves of the vegetation, the calculation target of the second vegetation index is not necessarily limited to the leaves. That is, vegetables such as bell peppers and chili peppers, fruit vegetables such as strawberries, or fruit tree crops may also be included in the calculation target of the second vegetation index.

In addition, when the first vegetation index 521 is acquired for each of the plurality of leaves and the number of first vegetation indices 521 is large, the number of second vegetation calculation standards generated by the calculation standard generation part 550 may increase. Then, since the second vegetation index calculation standard 551 may be finally generated by using the average value of the plurality of second vegetation index calculation standards, the accuracy of the second vegetation index calculation standard 551 may be increased.

In addition, the environmental data collection part 300 may include a soil environmental data collection part 310 and an atmospheric environmental data collection part 320.

The soil environmental data collection part 310 is configured to collect soil environmental data of the predetermined zone. The soil environmental data collection part 310 may collect the soil environmental data such as a soil color, a soil structure, and a soil moisture content by directly measuring the soil environmental data using a soil sensor or the like, or may collect the soil environmental data through wireless communication from the Forest Service or the like.

The atmospheric environmental data collection part 320 is configured to collect atmospheric environmental data of the predetermined zone. Furthermore, the atmospheric environmental data collection part 320 may collect the atmospheric environmental data such as a temperature and humidity, a precipitation amount, fine dust data, carbon dioxide data, or the like by directly measuring the atmospheric environmental data using an environmental sensor or the like, or may collect the atmospheric environmental data through wireless communication from the National Weather Service or the like.

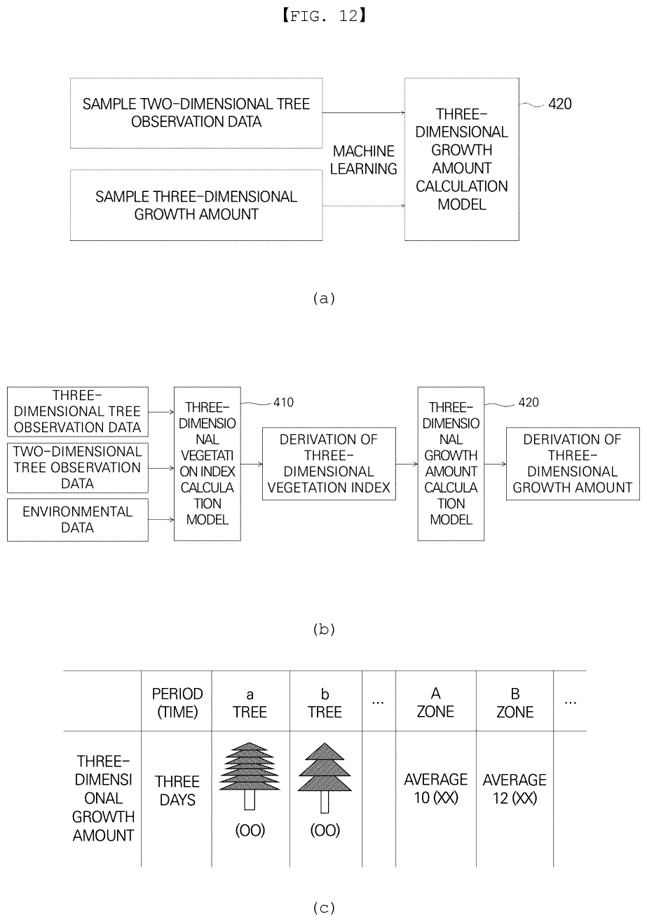

Referring to FIG. 10, the biological data prediction part 400 may include a three-dimensional vegetation index calculation part 410, a three-dimensional growth amount calculation part 420, a carbon dioxide capture amount calculation part 430, and a fine dust capture amount calculation part 440.

The three-dimensional vegetation index calculation part 410 is configured to calculate a three-dimensional vegetation index of trees. Furthermore, the three-dimensional vegetation index Calculation part 410 may receive sample three-dimensional tree observation data, sample two-dimensional tree observation data, sample environmental data, and a sample three-dimensional vegetation index, and may generate a three-dimensional vegetation index calculation model by performing machine learning matching the sample three-dimensional tree observation data, the sample two-dimensional tree observation data, the sample environmental data, and the sample three-dimensional vegetation index to each other. Furthermore, the three-dimensional vegetation index calculation model may calculate the three-dimensional vegetation index according to the three-dimensional tree observation data, the two-dimensional tree observation data, and the environmental data of the predetermined zone that are input into the three-dimensional vegetation index calculation model.

For example, referring to FIG. 11, as illustrated in FIG. 11(a), a three-dimensional image corresponding to a 3D model of the sample tree object is input as the sample three-dimensional tree observation data, a two-dimensional image corresponding to a vegetation index of the sample tree is input as the two-dimensional tree observation data, and sample environmental data such as fine dust data, carbon dioxide data, a moisture content at the time when the sample tree is observed is input. Then, the three-dimensional vegetation index calculation model may be generated through the machine learning. Furthermore, as illustrated in FIG. 11(b) and FIG. 11(c), the three-dimensional tree observation data, and the two-dimensional tree observation data, and the environmental data of the predetermined zone collected according to time series are input into the three-dimensional vegetation index calculation model. Furthermore, as illustrated in FIG. 11(d), the three-dimensional vegetation index of the predetermined zone may be calculated by the there-dimensional vegetation index calculation model.

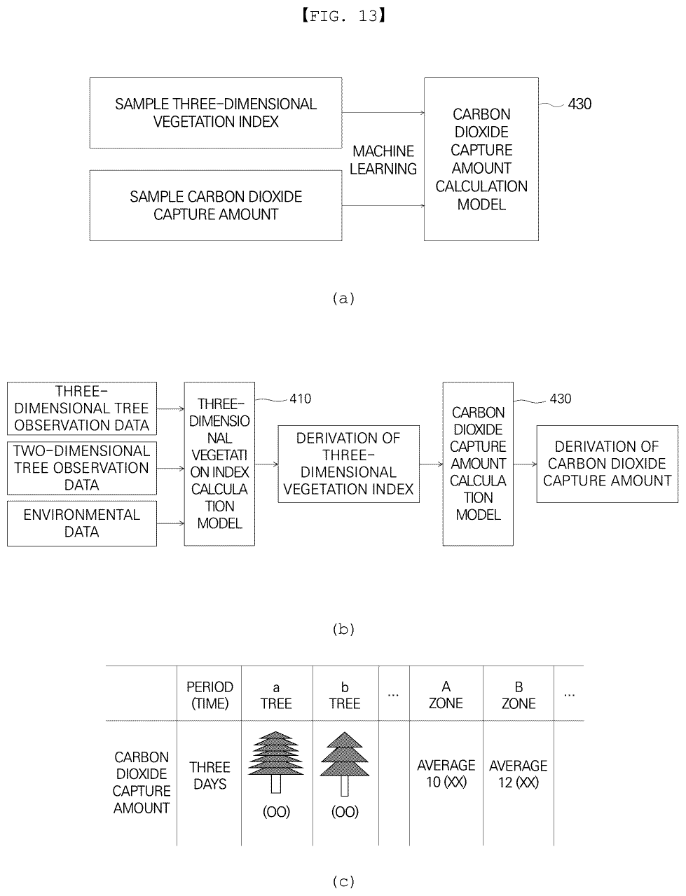

Then, referring to FIG. 12, the biological data prediction part 400 may include the three-dimensional growth amount calculation part 420.

The three-dimensional growth amount calculation part 420 is configured to calculate the three-dimensional growth amount of the trees. Furthermore, the three-dimensional growth amount calculation part 420 may receive a sample three-dimensional vegetation index and a sample three-dimensional growth amount, and may generate a three-dimensional growth amount calculation model by performing machine learning matching the sample three-dimensional vegetation index and the sample three-dimensional growth amount to each other. Furthermore, the three-dimensional growth amount calculation model may calculate the three-dimensional growth amount according to the input three-dimensional vegetation index of the trees in the predetermined zone.

For example, as illustrated in FIG. 12(a), when the sample three-dimensional vegetation index and the sample three-dimensional growth amount are input into the three-dimensional growth amount calculation part 420, the three-dimensional growth amount calculation model is generated by the machine learning matching the sample three-dimensional vegetation index with the sample three-dimensional growth amount. Furthermore, as illustrated in FIG. 12(b), when the three-dimensional image in the predetermined zone collected by the three-dimensional tree observation data collection part 100, the two-dimensional image in the predetermined zone collected by the two-dimensional tree observation data collection part 200, and the data on the predetermined zone collected by the environmental data collection part 300 are input into the three-dimensional vegetation index calculation model so as to calculate the three-dimensional vegetation index and then the three-dimensional vegetation index is input into the three-dimensional growth amount calculation model, the three-dimensional growth amount of the trees in the predetermined zone may be calculated as illustrated in FIG. 12(c).

Referring to FIG. 13, the biological data prediction part 400 may include the carbon dioxide capture amount calculation part 430.

The carbon dioxide capture amount calculation part 430 is configured to calculate the carbon dioxide capture amount of the trees. Furthermore, the carbon dioxide capture amount calculation part 430 may receive the sample three-dimensional vegetation index and a sample carbon dioxide capture amount, and may generate a carbon dioxide capture amount calculation model by performing machine learning matching the sample three-dimensional vegetation index and the sample carbon dioxide capture amount to each other. Furthermore, the carbon dioxide capture amount calculation model may calculate the carbon dioxide capture amount according to the input three-dimensional vegetation index in the predetermined zone.

For example, as illustrated in FIG. 13(a), when the sample three-dimensional vegetation index and the sample carbon dioxide capture amount are input into the carbon dioxide capture amount calculation part 430, the carbon dioxide capture amount calculation model is generated by the machine learning matching the sample three-dimensional vegetation index with the sample carbon dioxide capture amount. Furthermore, as illustrated in FIG. 13(b), when the three-dimensional image collected by the three-dimensional tree observation data collection part 100, the two-dimensional image collected by the two-dimensional tree observation data collection part 200, and the data collected by the environmental data collection part 300 are input into the three-dimensional vegetation index calculation model so as to calculate the three-dimensional vegetation index and then the three-dimensional vegetation index is input into the carbon dioxide capture amount calculation model, the carbon dioxide capture amount of the trees in the predetermined zone may be calculated as illustrated in FIG. 13(c).

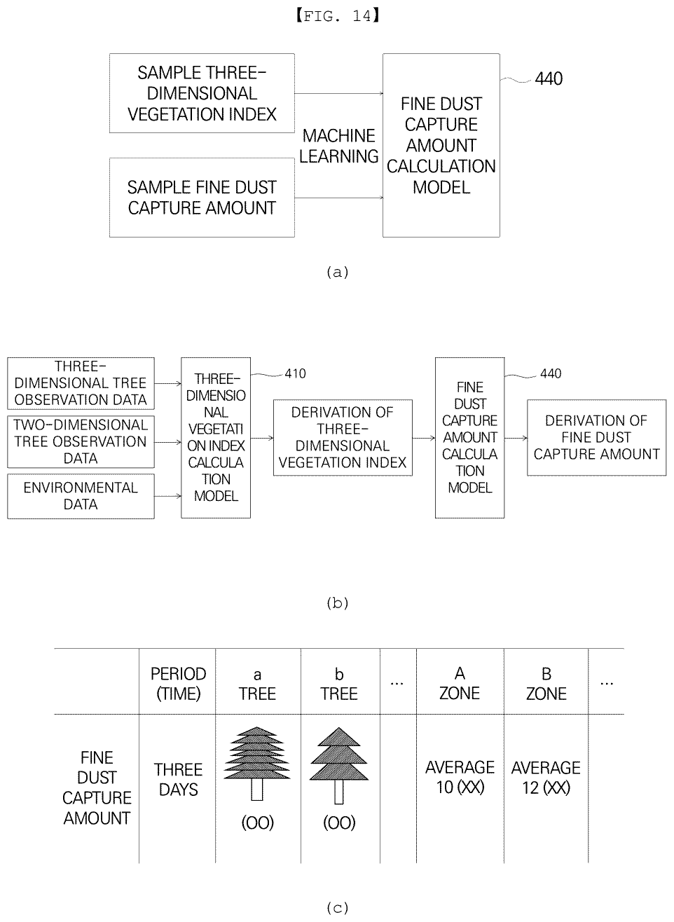

Referring to FIG. 14, the biological data prediction part 400 may include the fine dust capture amount calculation part 440.

The fine dust capture amount calculation part 440 is configured to calculate the fine dust capture amount of the trees. Furthermore, the fine dust capture amount calculation part 440 may receive the sample three-dimensional vegetation index and a sample fine dust capture amount, and may generate a fine dust capture amount calculation model by performing machine learning matching the sample three-dimensional vegetation index and the sample fine dust capture amount to each other. Furthermore, the fine dust capture amount calculation model may calculate the fine dust capture amount according to the input three-dimensional vegetation index in the predetermined zone.

For example, as illustrated in FIG. 14(a), when the sample three-dimensional vegetation index and the sample fine dust capture amount are input into the fine dust capture amount calculation part 440, the fine dust capture amount calculation model is generated by the machine learning matching the sample three-dimensional vegetation index with the sample fine dust capture amount. Furthermore, as illustrated in FIG. 14(b), when the three-dimensional image collected by the three-dimensional tree observation data collection part 100, the two-dimensional image collected by the two-dimensional tree observation data collection part 200, and the data collected by the environmental data collection part 300 are input into the three-dimensional vegetation index calculation model so as to calculate the three-dimensional vegetation index and then the three-dimensional vegetation index is input into the fine dust capture amount calculation model, the fine dust capture amount of the trees in the predetermined zone may be calculated as illustrated in FIG. 14(c).

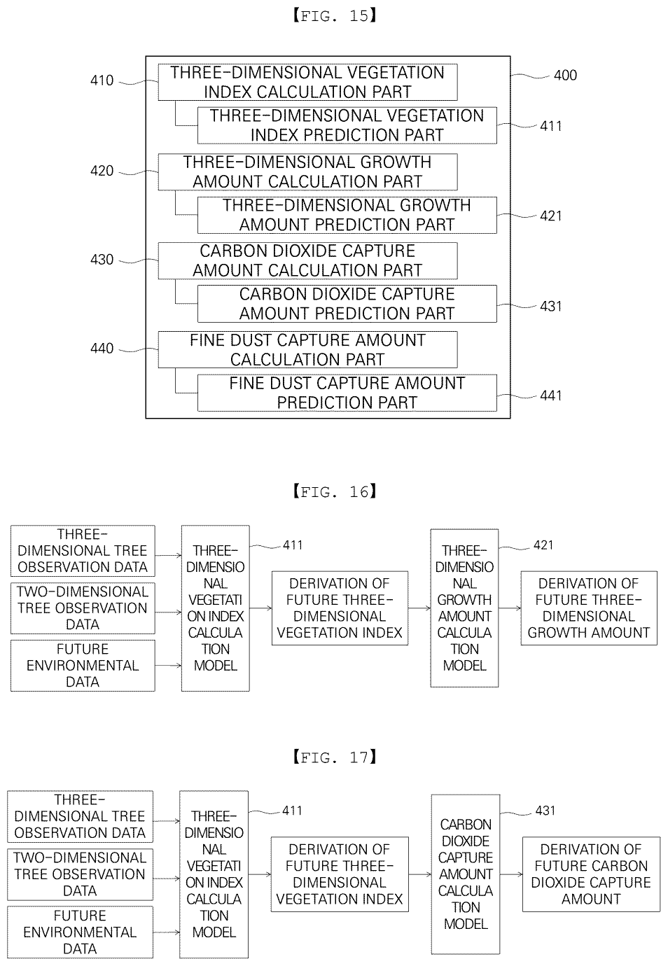

Referring to FIG. 15, the biological data prediction system for trees according to another embodiment of the present disclosure further includes a three-dimensional vegetation index prediction part 411, a three-dimensional growth amount prediction part 421, a carbon dioxide capture amount prediction part 431, and a fine dust capture amount prediction part 441.

First, the three-dimensional vegetation index prediction part 411 is configured to predict a future three-dimensional vegetation index of the trees in the predetermined zone from the three-dimensional vegetation index calculation model. Furthermore, when a three-dimensional image corresponding to the three-dimensional tree observation data of the predetermined zone, a two-dimensional image corresponding to the two-dimensional tree observation data of the predetermined zone, and future environmental data the predetermined zone collected from the Forest Service and the National Weather Service are input into the three-dimensional vegetation index calculation model, the future three-dimensional vegetation index of the predetermined zone may be predicted.

For example, when a tree species and an age of the trees in the predetermined zone are “pine tree/20 years”, a three-dimensional image which is a 3D model of a tree object corresponding to “pine tree/20 years” and which is collected by the three-dimensional tree observation data collection part 100, a two-dimensional image which is the two-dimensional tree observation data and which is a vegetation index of “pine tree/20 years”, and the future environmental data of the predetermined zone predicted from the Forest Service and the National Weather Service are input, so that the future three-dimensional vegetation index of the trees in the predetermined zone may be calculated by the three-dimensional vegetation index calculation model in which the machine learning is performed.

The three-dimensional growth amount prediction part 421 is configured to predict a future three-dimensional growth amount of the trees in the predetermined zone from the three-dimensional growth amount calculation model, and may predict the future three-dimensional growth amount of the trees in the predetermined zone only by the future three-dimensional vegetation index.

For example, referring to FIG. 16, in a situation in which the tree species and the age of the trees in the predetermined zone are “pine tree/20 years”, first, a three-dimensional image that is a 3D model of a tree object corresponding to “pine tree/20 years”, a two-dimensional image that is a vegetation index of “pine tree/20 years”, and the future environmental data of the predetermined zone predicted from the Forest Service and the National Weather Service are input by the three-dimensional vegetation index prediction part 411, a future three-dimensional vegetation index of the trees in the predetermined zone is calculated by the three-dimensional vegetation index calculation model. Furthermore, when the future three-dimensional vegetation index corresponding to “pine tree/20 years” is input into the three-dimensional growth amount prediction part 421, the future three-dimensional growth amount corresponding to “pine tree/20 years” in the predetermined zone may be calculated by the three-dimensional growth amount calculation model.

The carbon dioxide capture amount prediction part 431 is configured to predict a future carbon dioxide capture amount of the trees in the predetermined zone from the carbon dioxide capture amount calculation model, and may predict the future carbon dioxide capture amount in the predetermined zone only by the future three-dimensional vegetation index.

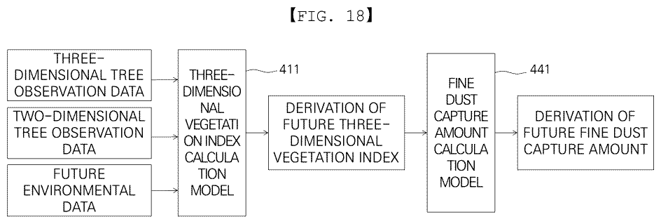

For example, referring to FIG. 17, in a situation in which the tree species and the age of the trees in the predetermined zone are “pine tree/20 years”, first, a three-dimensional image that is a 3D model of a tree object corresponding to “pine tree/20 years”, a two-dimensional image that is a vegetation index of “pine tree/20 years”, and the future environmental data of the predetermined zone predicted from the Forest Service and the National Weather Service are input by the three-dimensional vegetation index prediction part 411, a future three-dimensional vegetation index of the trees in the predetermined zone is calculated by the three-dimensional vegetation index calculation model. Furthermore, when the future three-dimensional vegetation index corresponding to “pine tree/20 years” is input into the carbon dioxide capture amount prediction part 431, the future carbon dioxide capture amount corresponding to “pine tree/20 years” in the predetermined zone may be calculated by the carbon dioxide capture amount calculation model.

The fine dust capture amount prediction part 441 is configured to predict a future fine dust capture amount of the trees in the predetermined zone from the fine dust capture amount calculation model, and may predict the future fine dust capture amount calculation amount in the predetermined zone only by the future three-dimensional vegetation index.

For example, referring to FIG. 18, in a situation in which the tree species and the age of the trees in the predetermined zone are “pine tree/20 years”, first, a three-dimensional image that is a 3D model of a tree object corresponding to “pine tree/20 years”, a two-dimensional image that is a vegetation index of “pine tree/20 years”, and the future environmental data of the predetermined zone predicted from the Forest Service and the National Weather Service are input by the three-dimensional vegetation index prediction part 411, a future three-dimensional vegetation index of the trees in the predetermined zone is calculated by the three-dimensional vegetation index calculation model. Furthermore, when the future three-dimensional vegetation index corresponding to “pine tree/20 years” is input into the fine dust capture amount prediction part 441, the future fine dust capture amount corresponding to “pine tree/20 years” in the predetermined zone may be calculated by the fine dust capture amount calculation model.

Although the present disclosure has been shown and described with reference to the preferred embodiments, the scope of the present disclosure is not limited thereto. Numerous other modifications and embodiments can be devised by those skilled in the art that will fall within the spirit and scope of the principles of the invention described in the appended claims. Furthermore, these modified embodiments should not be understood individually from the technical spirit or perspective of the present disclosure.

### INDUSTRIAL APPLICABILITY

The biological data prediction system for trees according to the present disclosure is capable of predicting biological data of the trees, such as a carbon dioxide capture amount, a fine dust capture amount, and so on, with a single physical quantity such as a vegetation index, so that the biological system is capable of being used in the fields of agriculture and forestry, or is capable of being used in a playground, a park, and so on that require management of a large number of trees and plants.

1. a biological data prediction system for trees, the biological data prediction system comprising:
a three-dimensional tree observation data collection part configured to collect three-dimensional tree observation data in a predetermined zone;
a two-dimensional tree observation data collection part configured to collect two-dimensional tree observation data in the predetermined zone;
an environmental data collection part configured to collect environmental data in the predetermined zone; and
a biological data prediction part configured to predict biological data of the trees from the three-dimensional tree observation data, the two-dimensional tree observation data, and the environmental data.

2. The biological data prediction system of claim 1, wherein the three-dimensional tree observation data collection part comprises at least one selected from:
a ground stationary three-dimensional measurement part fixed to a ground and configured to collect three-dimensional tree observation data in the predetermined zone while being in a stationary state;
an aerial mobile three-dimensional measurement part disposed on an aerial portion of the predetermined zone such that the aerial mobile three-dimensional measurement part is in a mobile state, the aerial mobile three-dimensional measurement part being configured to collect three-dimensional tree observation data in the aerial portion of the predetermined zone while being in the mobile state; and
a ground mobile three-dimensional measurement part disposed on the ground of the predetermined zone such that the ground mobile three-dimensional measurement part is in the mobile state, the ground mobile three-dimensional measurement part being configured to collect three-dimensional tree observation data in the predetermined zone while being in the mobile state.

3. The biological data prediction system of claim 1, wherein the two-dimensional tree observation data collection part comprises a multispectral camera.

4. The biological data prediction system of claim 1, wherein the environmental data collection part comprises:
a soil environmental data collection part configured to collect soil environmental data in the predetermined zone; and
an atmospheric environmental data collection part configured to atmospheric environmental data in the predetermined zone.

5. The biological data prediction system of claim 1, wherein the biological data prediction part comprises a three-dimensional vegetation index calculation part configured to calculate a three-dimensional vegetation index the trees in the predetermined zone.

6. The biological data prediction system of claim 5, wherein the biological data prediction part further comprises a three-dimensional growth amount calculation part configured to calculate a three-dimensional growth amount of the trees in the predetermined zone.

7. The biological data prediction system of claim 5, wherein the biological data prediction part further comprises a carbon dioxide capture amount calculation part configured to calculate a carbon dioxide capture amount of the trees in the predetermined zone.

8. The biological data prediction system of claim 5, wherein the biological data prediction part further comprises a fine dust capture amount calculation part configured to calculate a fine dust capture amount of the trees in the predetermined zone.
